(19) United States
(12) Patent Application Publication
(10) Pub. No.: US 2012/0150309 A1
(43) Pub. Date: Jun. 14, 2012

(54) HINGE STRUCTURE

(76) Inventor: Roelof Marissen, Born (NL)

(21) Appl. No.: 13/318,220

(22) PCT Filed: Apr. 28, 2010
(86) PCT No.: PCT/EP2010/055808

§ 371 Date: Feb. 27, 2012

(30) Foreign Application Priority Data
Apr. 29, 2009 (EP) 09159023.2

(51) Int. Cl. A61F 2/42; B65D 43/16; E05D 7/00

(52) U.S. Cl. 623/21.15; 16/385; 220/810

(57) ABSTRACT

The invention relates to a hinge comprising a first rigid body, a second rigid body and a joint member connected to the first rigid body and the second rigid body allowing substantially two-dimensional rotation of the first rigid body and the second and rigid body relative to each other. The joint member comprises HPPE fibers which are bendable by the rotation.

The invention relates to a hinge and in particular to a hinge comprising polyethylene. The invention also relates to devices comprising such a hinge, such as a medical device comprising such a hinge, in particular to an artificial finger joint comprising such a hinge.

Medical products such as artificial joints typically utilize a combination of polymer and metallic alloys. The metallic alloys are employed for the articulation and the polymer is employed as the soft socket. The polymer part wears against the metal articulation part during use, and ultrafine particles of polymer are loosened into the body over time. These particles are known to cause resorption of the periprosthetic bone. This results in loosening of the prosthesis components, possibly requiring even revision surgery.

WO2007/002409 and US2007016193 disclose an implant device for flexibly linking at least two vertebra of a spine of a patient. The device comprises two rods connected by a partially folded segment. The two rods are connected to the podicle screws, which in turn are connected to the vertebra. At least part of the device may be comprised of the group consisting of: titanium, polyether ether ketone, polyether ketone ketone, ultra high molecular weight polyethylene, and polymethylmethacrylate (PMMA). Such a device is repeatedly bent during use and needs to have a strong resistance against bend fatigue. There is a need in the industry for a device with an improved bend fatigue resistance.

The object of the present invention is to provide a hinge with improved mechanical properties.

The object is achieved according to the invention by a hinge comprising a first rigid body, a second rigid body and a joint member connected to the first rigid body and the second rigid body allowing substantially two-dimensional rotation of the first rigid body and the second rigid body relative to each other, wherein the joint member comprises high performance polyethylene (HPPE) fibers which are bendable by the rotation.

According to the present invention, the HPPE fibers in the joint member is bent by the movement of the first (second) rigid body with respect to the second (first) rigid body. The first rigid body and the second rigid body rotate relative to each other about a rotational axis. The rotation may preferably limited within a certain fixed angle. The angle of rotation may be limited by any suitable means. The dimension and the construction of the joint member may be chosen with respect to the distance between the first and the second rigid bodies to allow rotation within only a fixed angle. The first and the second rigid bodies may simply contact each other at a fixed angle to prevent further rotation. The joint member itself may have a resilience which allows deformation only to a certain degree. The skilled person knows how to limit the rotation angle and therefore it is not described herein in detail.

It should be observed that the ultra high molecular weight polyethylene suggested in WO2007/002409 and US2007016193 solely refer to particulate powder. There is no indication, teaching or suggestion in the disclosure that the ultra high molecular weight polyethylene starting material could be in any other configuration than particulate powder. For example, the only fibrous material described is carbon fiber composites. UHMWPE powder is not a HPPE fiber. Particularly, the properties and processing is completely different.

The HPPE fibers in the joint member show a very high resistance against bend fatigue. This is extremely advantageous since the most important function of a hinge is to be able to be bent numerous times without breaking.

Within the context of the present invention, fibres are understood to mean elongated bodies of indefinite length and with length dimension much greater than width and thickness. The term fibre thus includes a monofilament, a multifilament yarn, a ribbon, a strip or tape and the like, and can have regular or irregular cross-section. The term fibres also includes a plurality of any one or combination of the above.

Fibres having the form of monofilaments or tape-like fibres can be of varying titer, but typically have a titer in the range of 10 to several thousand dtex, preferably in the range of 100 to 2500 dtex, more preferably 200-2000 dtex. Multi-filament yarns contain a plurality of filaments each having a titer typically in the 0.2-25 dtex range, preferably about 0.5-20 dtex. The titer of a multifilament yarn may also vary widely, for example from 10 to several thousand dtex, but is preferably in the range of about 200-4000 dtex, more preferably 300-3000 dtex. HPPE fibres are herein understood to be fibres made from polyethylene and having a tenacity of at least 1.5 N/tex, preferably at least 2.0 N/tex, more preferably at least 2.5 N/tex or even at least 3.0 N/tex. Tensile strength, also simply strength, or tenacity of fibres are determined by known methods, as based on ASTM D885-85 or D2256-97. There is no reason for an upper limit of tenacity of HPPE fibres, but available fibres typically are of tenacity at most about 5 to 6 N/tex. The HPPE fibres also have a high tensile modulus, e.g. of at least 75 N/tex, preferably at least 100 or at least 125 N/tex. HPPE fibres are also referred to as high-modulus polyethylene fibres.

HPPE fibres can be prepared by spinning of a solution of ultrahigh molecular weight polyethylene (UHMWPE) in a suitable solvent into gel fibres and drawing the fibres before, during and/or after partial or complete removal of the solvent; that is via a so-called gel-spinning process. Gel spinning of a solution of UHMWPE is well known to the skilled person; and is described in numerous publications, including EP 0205960 A, EP 0213208 A1, U.S. Pat. No. 4,413,110, GB 2042414 A, EP 0200547 B1, EP 0472114 B1, WO 01/73173 A1, and in Advanced Fiber Spinning Technology, Ed. T. Nakajima, Woodhead Publ. Ltd (1994), ISBN 1-855-73182-7, and in references cited therein, all incorporated herein by reference.

UHMWPE is understood to be polyethylene having an intrinsic viscosity (IV, as measured on solution in decalin at 135° C.) of at least 5 dl/g, preferably of between about 8 and 40 dl/g. Intrinsic viscosity is a measure for molar mass (also called molecular weight) that can more easily be determined than actual molar mass parameters like Mₙ and M_{w}. There are several empirical relations between IV and M_{w}, but such relation is dependent on molar mass distribution. Based on the equation M_{w}=5.37*10⁴ [IV]^{1.37} (see EP 0504954 A1) an IV of 8 dl/g would be equivalent to M_{w} of about 930 kg/mol. Preferably, the UHMWPE is a linear polyethylene with less than one branch per 100 carbon atoms, and preferably less than one branch per 300 carbon atoms; a branch or side chain or chain branch usually containing at least 10 carbon atoms. The linear polyethylene may further contain up to 5 mol % of one or more comonomers, such as alkenes like propylene, butene, pentene, 4-methylpentene or octene.

When HPPE fibers are prepared by the gel-spinning process, the residual solvent content is preferably at most 100 ppm. This is especially important when the hinge is used in medical applications. The residual solvent content is herein understood to mean the content of the solvent used in making the HPPE fibers, which solvent is still remaining in the final fibers. In the process of making the yarn, any of the known solvents for gel spinning of UHMwPE can be used.

HPPE fibers can also be prepared by melt-spinning of polyethylene having a high molecular weight, although the mechanical properties such as tenacity are more limited compared to HPPE fibres made by the gel-spinning process. The upper limit of the molecular weight of the polyethylene which can be melt-spun can be in the range of the molecular weight of UHMWPE, but is lower than the limit with the gel-spinning process. Melt-spun HPPE fibers can also be prepared from polyethylene having a molecular weight lower than UHMWPE as defined above. The melt-spinning process is widely known in the art, and involves heating a PE composition to form a PE melt, extruding the PE melt, cooling the extruded melt to obtain a solidified PE, and drawing the solidified PE at least once. The process is mentioned e.g. in EP1445356A1 and EP1743659A1, which are incorporated herein by reference.

In one embodiment, the UHMWPE contains a small amount, preferably at least 0.2, or at least 0.3 per 1000 carbon atoms, of relatively small groups as pending side groups, preferably a C1-C4 alkyl group. Such a fibre shows an advantageous combination of high strength and creep resistance. Too large a side group, or too high an amount of side groups, however, negatively affects the process of making fibres. For this reason, the UHMWPE preferably contains methyl or ethyl side groups, more preferably methyl side groups. The amount of side groups is preferably at most 20, more preferably at most 10, 5 or at most 3 per 1000 carbon atoms.

The HPPE fibres may further contain small amounts, generally less than 5 mass %, preferably less than 3 mass % of customary additives, such as anti-oxidants, thermal stabilizers, colorants, flow promoters, etc. The UHMWPE can be a single polymer grade, but also a mixture of two or more different polyethylene grades, e.g. differing in IV or molar mass distribution, and/or type and number of comonomers or side groups.

The hinge according to the present invention does not require a construction of a typical conventional hinge with a joint comprising two members each having a specially adapted shape for engaging with each other. Typically the joint of a conventional hinge comprises a first member having a cylindrical portion and a second member having a tube portion, wherein the cylindrical portion is received inside the tube portion, and the cylindrical portion rotates around a rotational axis at the center of the tube portion. Unlike the conventional hinges, the joint member of the hinge according to the present invention may consist of one integral body which is bendable by the rotation, which gives an important advantage that the hinge has a much simpler construction. By the joint member being one integral body, it is herein meant that the joint member consists of one non-separable element which is bent for allowing rotation.

The first and the second rigid bodies may be made of any suitable materials, including metal, ceramic and polymer and a combination thereof. Non-metal material is preferred for embodiments where the rigid body contacts with the HPPE fibers of the joint member by the rotational movement of the rigid bodies. This is in view of the additional wear of the joint member caused by the rigid body making abrasive contact with the joint member. Furthermore, the rotational movement of the rigid bodies according to the invention leads to a large and controllable range of motion of the rigid bodies.

Preferably, the first rigid body and/or the second rigid body comprises compression moulded HPPE fibers. Preferably, at least part of the compression moulded HPPE fibers are positioned at the surface of the rigid body. By compression moulded, it is herein meant that HPPE fibers arranged in a certain shape and have been subjected to an elevated pressure at an elevated temperature to be at least partially melted together to form one body. Preferably, the pressure and the temperature are chosen in a range where the mechanical properties such as tenacity of the HPPE fibers do not deteriorate substantially. This embodiment has an advantage that the rigid bodies have a high abrasion resistance. Also this embodiment has an important advantage that the rigid bodies may be made without using any material that may be detrimental for use in medical applications.

According to one embodiment of the present invention, the joint member includes a fabric comprising the HPPE fibers. By a fabric, which in the art is also called textile or cloth, is herein understood a sheet-like structure comprising interlaced filaments or yarns, said fabric having a thickness much smaller than its other two dimensions, i.e. the axial dimension and the transversal dimension. The fabric of the invention may be of any construction known in the art, e.g. woven, knitted, plaited, braided or non-woven or combinations thereof.

In a further embodiment, the joint member includes a yarn comprising the HPPE fibers. By a yarn is herein meant any elongated body with its transversal dimension much smaller than its length made from fibers by twisting, twining, braiding or the like, and includes a cable or a braid.

According to one embodiment of the present invention, the fabric or the yarn made of the HPPE fibers is directly connected to the first and the second rigid bodies. This may be done e.g. by embedding part of the fabric in the first and the second rigid bodies. According to one preferred embodiment of the present invention, at least some of the HPPE fibers extend into the first rigid body and/or the second rigid body. The HPPE fibers in the joint member extending into the rigid body provide a strong connection between the joint member and the rigid body.

Alternatively, the HPPE fibers may be used only in the bendable portion of the joint member. This may be advantageous from a cost point of view. The joint member may contain additional elements than the HPPE fibers, which are connected to the first and the second rigid bodies.

Preferably, at least some of the fibers are arranged in a generally perpendicular orientation with respect to the rotational axis. These fibers give an extra resistance against bend fatigue.

In a preferred embodiment, the first rigid body and/or the second rigid body comprise the HPPE fibers impregnated in a resin. Such an embodiment has an advantage that it may be made in a very simple manner. Fibers may be arranged randomly or in a specific orientation or construction, and a resin may be added e.g. by simply pouring the resin onto the fibers. It will be appreciated that also fibers may be added to the resin and the order is not crucial in this embodiment. Various types of composite production processes can be used as known to the skilled person. After curing, the resin may be bent at a line intersecting at least one HPPE fiber where the rotational axis of the hinge is to be made. After the bending or a multiple times of the bending, the resin will eventually break. The breaking of the resin does not result in a HPPE fiber breakage, and the joint member is provided by the bendable HPPE fibers. The resin broken into two parts provides the two rigid bodies. Alternatively, the part of the fiber arrangement intended to be the joint member may be excluded from applying the resin. In this case, the bending step is not required and the manufacturing process may be simpler. Resin is preferably chosen from the biocompatible type of resins. Multitude of geometries having fibrous connections between two rigid bodies is possible.

Making a rigid body from HPPE fibers without using a resin is also possible. This can be done e.g. by compression moulding of the HPPE fibers as mentioned above. HPPE fibers may be arranged into a desired shape, e.g. a plate-like member, and the plate-like member may be consolidated except for one segment extending between opposite edges. The consolidated parts on both sides of the segment provide the first body and the second rigid body, and the non-consolidated segment provides the joint member.

In a further embodiment, the joint member comprises a yarn formed from the HPPE fibers and the yarn connects the first rigid body and the second rigid body so as to allow the rotation relative to each other.

The yarn may be attached to the first and/or the second rigid body by a knot made from the yarn. This may be implemented in a number of ways. For example, the rigid body may be provided with a hole through which the yarn penetrates, and a knot is made to prevent the yarn from being removed from the rigid body. For example, by making a knot larger than the diameter of the hole after the yarn penetrated through the hole, the yarn is prevented from being removed from the rigid body with the hole. Both the first and the second rigid bodies may be provided with a hole, and a loop may be formed from the yarn penetrating through the two holes. Preferably, each of the rigid body is provided with at least two holes through which the yarn penetrates. This allows limiting the axis of the rotational movement of the rigid bodies.

In another embodiment, the first and/or the second rigid bodies comprise a protrusion around which a knot is formed. In a particularly preferred embodiment, the first and the second rigid bodies have substantially T-shape at one end, the first and the second rigid bodies arranged in such a way that the head portions of the T-shape are substantially parallel, wherein the yarn is wound around the head portions to allow rotation around the rotational axis between the head portions of the first and the second rigid bodies.

In another preferred embodiment, each of the first rigid body and the second rigid body has a substantially sheet-like portion. Preferably, the first rigid body and the second rigid body have a sheet-like shape. The sheet-like portion has a first face and a second face opposite the first face. The first face of the first rigid body and the first face of the second rigid body are on the same side. The joint member comprises at least a first member and a second member. The first member and the second member may be connected or separate. The first member extends from the first side of the first rigid body to the second side of the second rigid body. The second member extends from the second side of the first rigid body to the first side of the second rigid body. It will be appreciated that the joint member may comprise further members extending from one side from the first rigid body to the other side of the second rigid body.

The present invention also relates to a medical device comprising a hinge according to the present invention. Preferably, the joint member substantially consists of the HPPE fibers. Preferably, the first and the second rigid bodies substantially consist of biocompatible materials, such as a surgical stainless steel, or a polymer, preferably a polyolefin, more preferably polyethylene, most preferably a powder based or a fiber based UHMWPE. In the most preferred embodiment, the medical device substantially consists of HPPE fibers. For example, the first and the second rigid bodies may be made from compression moulded HPPE fibers. These rigid bodies may then be connected by HPPE fibers. Alternatively, HPPE fibers may be arranged into a desired shape and those parts intended to be the rigid bodies may be consolidated by e.g. compression moulding. The remaining part will maintain its flexibility and provide the joint member.

The present invention also relates to an artificial joint comprising a hinge according to the present invention.

The present invention also relates to an artificial finger joint comprising a hinge according to the present invention. Preferably, the first and the second rigid bodies of the artificial finger joint have substantially T-shape. The first and the second rigid bodies are arranged in such a way that the head portions of the T-shape are substantially parallel. Yarn formed from the HPPE fibers is wound around the head portions to allow rotation around a rotational axis between the head portions of the first and the second rigid bodies.

The hinge according to the present invention can be used for various mechanical applications involving repeated bending. For example, a robot arm comprising the hinge according to the present invention is especially advantageous. The ability to allow repeated bending particularly in combination with the low wear due to the rotational movement of the rigid bodies is a major advantage of the present invention. It should be observed that the wear remains low even at high pressure of the hinge construction. Particularly in medical applications wear and associated production of wear particles is a major concern, which may be solved by the present invention.

The present invention also relates to a container comprising the hinge according to the invention. The container comprises a containing body and a door movable with respect to the containing body by means of the hinge. The containing body and the door comprise the first rigid body and the second rigid body, respectively. Preferably, the first rigid body and the second rigid body comprise the HPPE fibers impregnated in a resin. In a particularly advantageous embodiment, the container is an air freight container. The air freight container may be made with one integral body made of a network of HPPE fibers consolidated in a suitable way. This is especially advantageous in that the separate hinge used in conventional air freight container is not necessary, leading to the construction of the air freight container being more simple and robust.

The invention is hereinafter further illustrated with reference to the attached drawings in which:

FIG. 1 is a diagrammatic perspective view of an embodiment of the hinge of the present invention;

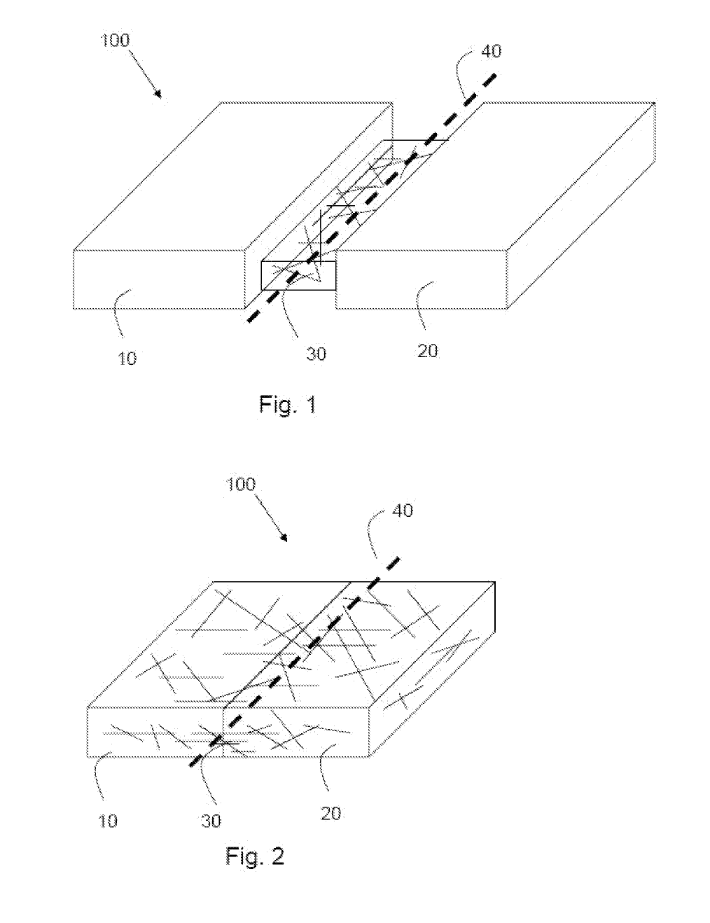

FIG. 2 is a diagrammatic perspective view of a further embodiment of the hinge of the present invention;

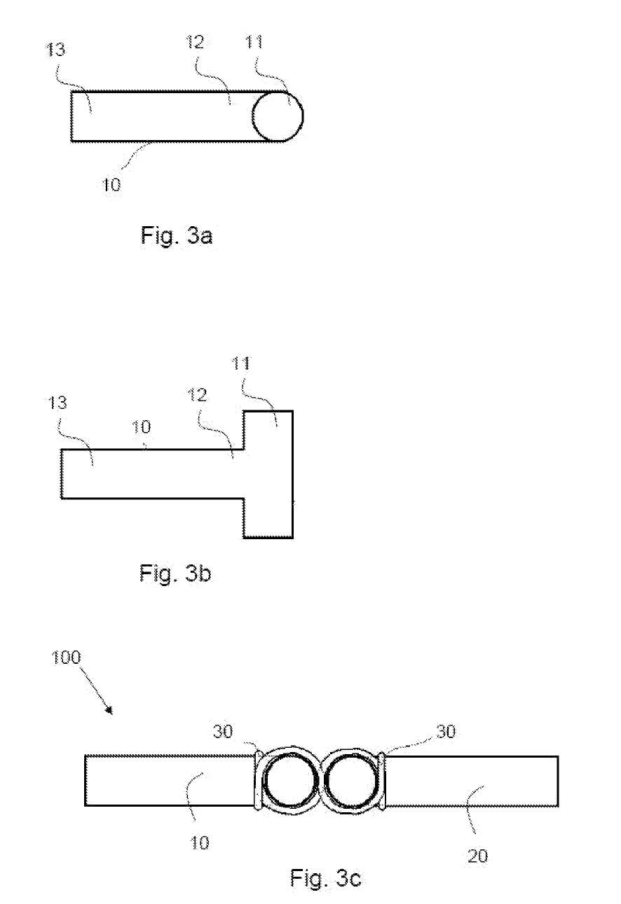

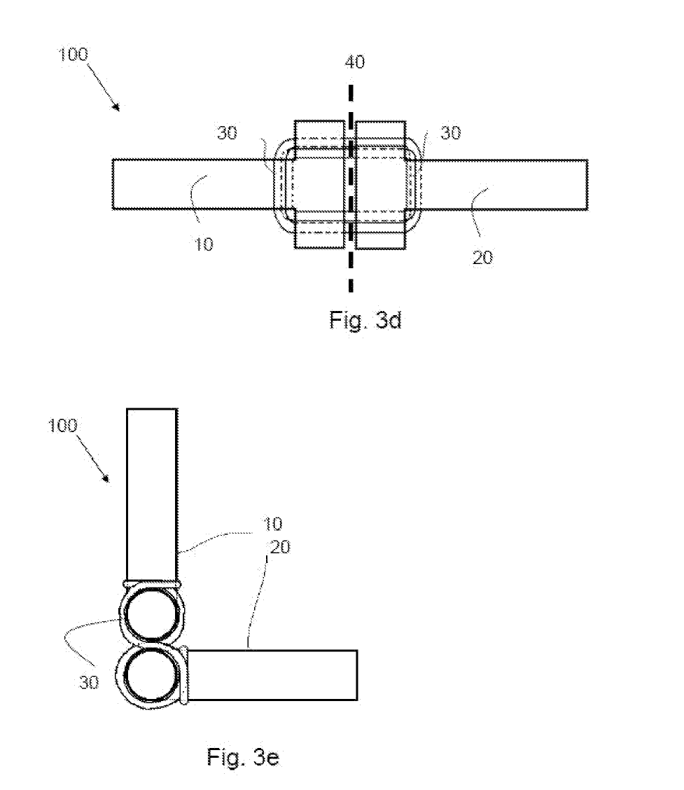

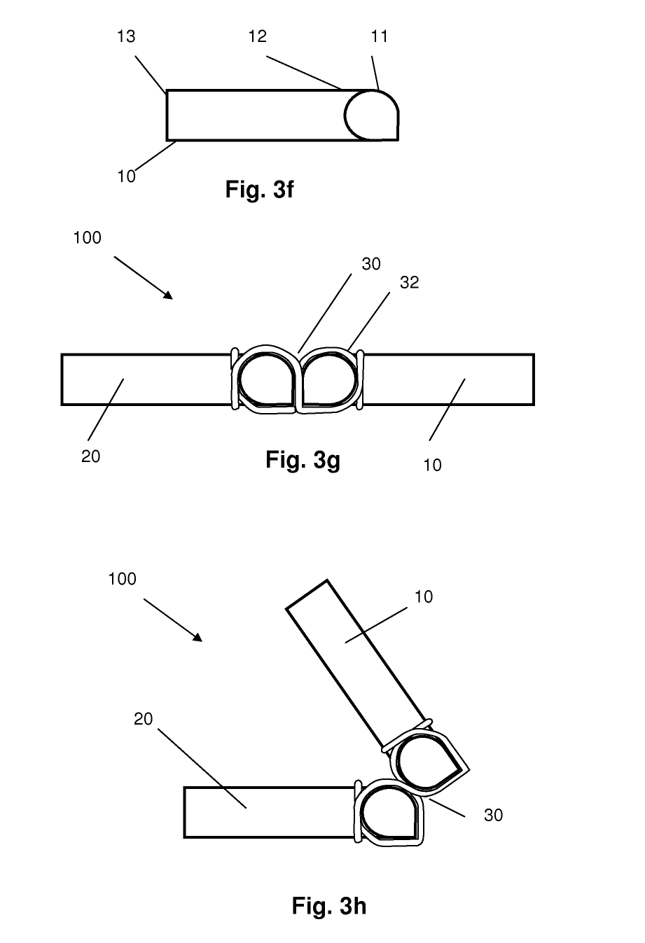

FIGS. 3a and 3b show an embodiment of a rigid body of the hinge according to the present invention;

FIGS. 3c-3e show a further embodiment of the hinge of the present invention;

FIGS. 3f-3h shows a further embodiment of a rigid body and a hinge with controlled movement according to the present invention;

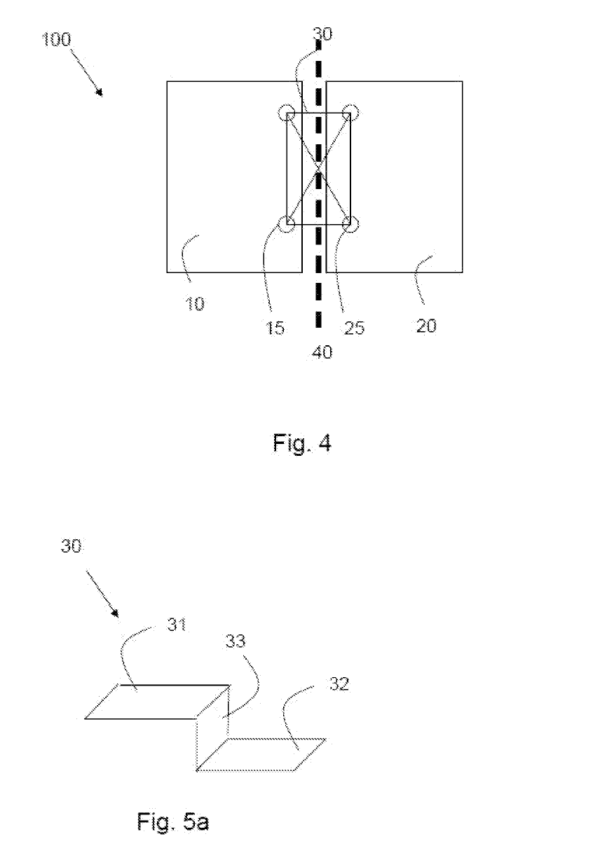

FIG. 4 diagrammatically shows a top view of a further embodiment of the hinge of the present invention;

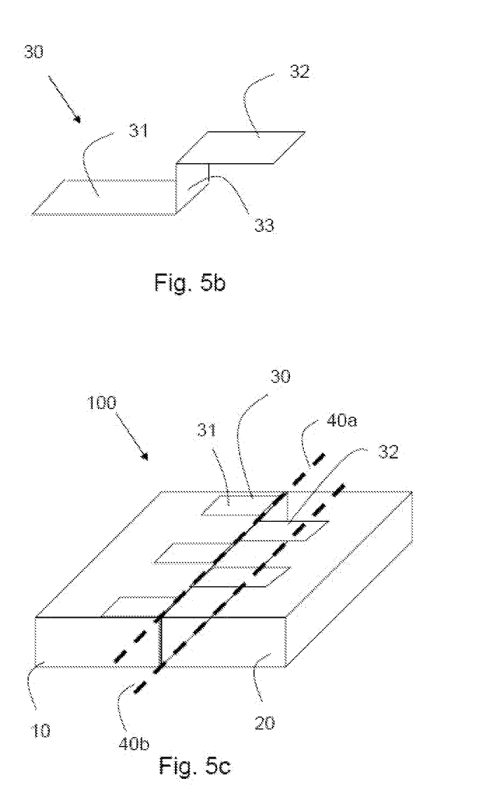

FIGS. 5a and 5b is a diagrammatic perspective view of an embodiment of the joint member of the hinge of the present invention.

FIG. 5c is a diagrammatic perspective view of a further embodiment of the hinge of the present invention and

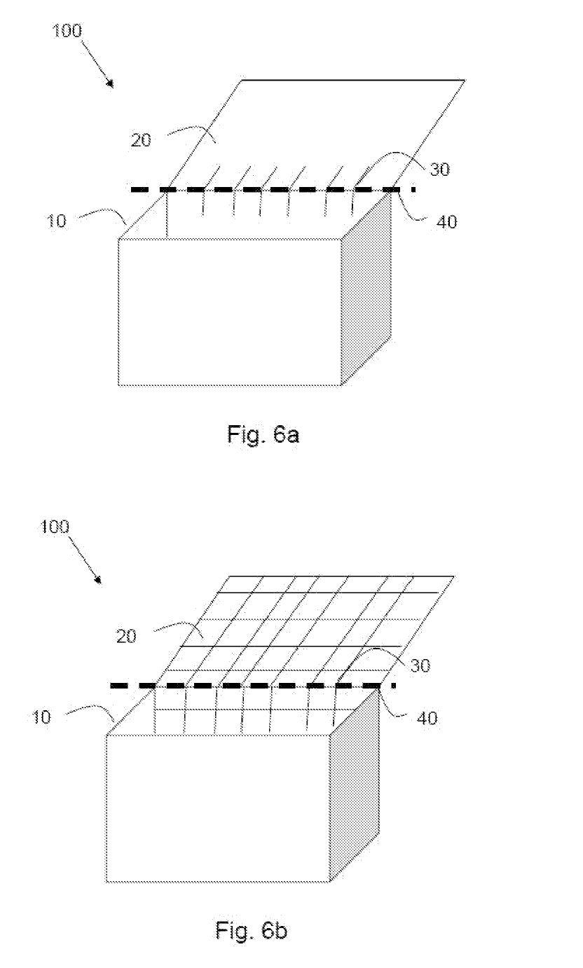

FIGS. 6a and 6b show an embodiment of a container of the present invention.

It is noted that the same reference numbers have been used for corresponding elements in the embodiments.

Referring to FIG. 1, a perspective view of an embodiment of a hinge 100 according to the present invention is shown. The hinge 100 comprises a first rigid body 10 and a second rigid body 20. A joint member 30 connects the first and the second rigid bodies 10 and 20. In this embodiment, the joint member 30 is a flexible sheet-like material in the form of a fabric. A rotational axis 40 is defined extending substantially parallel to the plane surface of the joint member 30 between the first rigid body 10 and the second rigid body 20. The joint member 30 comprises HPPE fibers which have been made into an integral structure by knitting, weaving or like. In this embodiment, the HPPE fibers are embedded in another flexible body which keeps the integral structure intact and protects the HPPE fibers from damage. The flexible body of the joint member 30 is connected to the first and the second rigid bodies 10 and 20.

It will be appreciated that a modification to this embodiment is possible in which the joint member substantially consists of a network of the HPPE fibers, i.e. the HPPE fibers are not embedded in a separate body but are directly connected to the first and the second rigid bodies 10 and 20. In such a case, the HPPE fibers preferably extend into the first and/or the second rigid bodies 10 and 20, and more preferably in both the first and the second rigid bodies 10 and 20.

FIG. 2 diagrammatically illustrates a perspective view of a further embodiment of a hinge 100 according to the present invention. A first rigid body 10 and a second rigid body 20 of the hinge 100 are made of e.g. a resin. HPPE fibers are embedded in the first and the second rigid bodies 10 and 20. Along a rotational axis 40 the resin has been broken to define the boundary between the first and the second rigid bodies 10 and 20. The network of the HPPE fibers provides the joint member 30.

FIGS. 3a and 3b illustrate a rigid body 10 to be used in the hinge 100 according to the present invention as illustrated in FIGS. 3c-3e. FIGS. 3a and 3b show a side view and a top view of the rigid body 10, respectively. The rigid body 10 is substantially T-shaped from the top view. The rigid body comprises a head portion 11 connected via a neck portion 12 to a body portion 13. FIG. 3c shows a side view of a hinge 100 comprising a rigid body 10 as shown in FIGS. 3a and 3b connected to a rigid body 20 having substantially the same shape as the rigid body 10. The connection is made by a joint member 30 which comprises a yarn or anther member (such as a braid) made of HPPE fibers. The joint member 30 is wound around the head portion 11 of the rigid body 10 and the head portion 21 of the rigid body 20. FIG. 3d shows a top view of the hinge 100. The winding of the joint member 30 allows a rotation around a rotational axis 40. FIG. 3e shows a side view of the hinge 100 in a different rotational position from FIG. 3c.

In many applications it is advantageous to control the range of motion (rotation) of the hinge as describe elsewhere in the present document. FIGS. 3f shows one embodiment of a rigid body 10, where the range of motion is limited. As the rigid body 10 in FIG. 3a, the body is preferably T-shaped (as shown in FIG. 3b). The body has a head portion 11, a neck portion 12 and a body portion 13. When arranged in a hinge configuration as shown in FIG. 3g, the rigid bodies 10 and 20 are aligned with the hooked part of the head towards each other. A HPPE fiber member 32, such as a yarn or a braid comprising HPPE fibers around and between the rigid bodies in a number 8-like pattern to keep the relationship between the rigid bodies 10 and 20. In this way, the rigid bodies 10 and 20 can not rotate in the direction corresponding to the body parts moving downwards but may easily move upwards as shown in FIG. 3h. In the shown version of the hinge 100 in FIGS. 3g and 3h, the rotation is basically limited to 180°. Such a configuration is very useful for example for a finger joint. The skilled person will appreciate that by changing the shape of the head, the range of motion may easily be adjusted accordingly for example to 0 to 90° or −45 to +45°.

FIG. 4 shows a top view of a further embodiment of the hinge 100 according to the present invention. The first and the second rigid bodies 10 and 20 comprise holes 15 and 25, respectively. A yarn or other body made of HPPE fibers connects the holes 15 and 25 and forms a joint member 30 in such a way that rotation around a rotational axis 40 is allowed.

FIGS. 5a and 5b are a diagrammatic perspective view of an embodiment of the joint member of the hinge of the present invention. The joint member 30 comprising HPPE fibers is a sheet-like material comprising a first portion 31, a second portion 32 and a middle portion 33 connecting the first portion 31 and the second portion 32. The first portion 31 and the second portion 32 are bendable with respect to the middle portion 33, and the joint member 30 may switch between the configurations shown in FIGS. 5a and 5b by the bending. FIG. 5c is a diagrammatic perspective view of a further embodiment of the hinge 100 of the present invention comprising three joint members 30 having the configuration shown in FIG. 5a and two joint members 30 having the configuration shown in FIG. 5b. The first rigid body 10 and the second rigid body 20 are both shaped as a sheet-like material having a first face and a second face facing opposite the first face. The middle portions 33 of the joint members 30 are sandwiched between the edges of the first and the second rigid bodies 10 and 20. In three of the joint members 30, the first portion 31 extends over a part of the first face of the first rigid body 10 and the second portion 32 extends over a part of the second face of the second rigid body 10. In two of the joint members 30, the first portion 31 extends over a part of the second face of the first rigid body 10 and the second portion 32 extends over a part of the first face of the second rigid body 10. The first rigid body 10 and the second rigid body 20 are thereby allowed to rotate with respect to each other. The second rigid body 20 is allowed to rotate in the anti-clockwise direction around a rotational axis 40a and in the clockwise direction around a rotational axis 40b.

FIGS. 6a and 6b show an embodiment of a container of the present invention. FIGS. 6a and 6b show a hinge 100 in the form of a container comprising a containing body 10 and a door 20. In FIG. 6a, the containing body 10 is connected to the door 20 by means of a joint member 30 in the form of HPPE fibers. In FIG. 6b, the containing body 10 and the door 20 consist of a network of HPPE fibers impregnated in a resin. Similarly to the embodiment described with respect to FIG. 2, the resin has been broken to define the boundary between the first and the second rigid bodies 10 and 20 to provide a rotational axis 40 of the door 20 with respect to the containing body 10. The network of the HPPE fibers provides the joint member 30.

1. A hinge comprising a first rigid body, a second rigid body and a joint member connected to the first rigid body and the second rigid body allowing substantially two-dimensional rotation of the first rigid body and the second rigid body relative to each other, wherein the joint member comprises HPPE fibers which are bendable by the rotation.

2. The hinge according to claim 1, wherein the joint member consists of an integral body.

3. The hinge according to claim 1, wherein the joint member includes a fabric comprising the HPPE fibers.

4. The hinge according to claim 1, wherein the joint member includes a yarn comprising the HPPE fibers,

5. The hinge according to claim 1, wherein at least some of the HPPE fibers extend into the first rigid body and/or the second rigid body.

6. The hinge according to claim 1, wherein the first rigid body and/or the second rigid body comprises the HPPE fibers impregnated in a resin.

7. The hinge according to claim 4, wherein the yarn connects the first rigid body and the second rigid body so as to allow the rotation relative to each other.

8. The hinge according to claim 7, wherein the yarn is attached to the first and/or the second rigid body by a knot made from the yarn.

9. The hinge according to claim 7, wherein the first and/or the second rigid body are provided with a hole through which the yarn penetrates and forms the knot.

10. The hinge according to claim 7, wherein the first and the second rigid bodies have substantially T-shape at one end, the first and the second rigid bodies arranged in such a way that the head portions of the T-shape are substantially parallel, wherein the yarn is wound around the head portions to allow rotation around a rotational axis between the head portions of the first and the second rigid bodies.

11. The hinge according to claim 1, wherein each of the first rigid body and the second rigid body has a substantially sheet-like portion having a first face and a second, opposite face, the first face of the first rigid body and the first face of the second rigid body being on the same side, and the joint member comprises a first member extending from the first side of the first rigid body to the second side of the second rigid body and a second member extending from the second side of the first rigid body to the first side of the second rigid body.

12. A medical device comprising the hinge according to claim 1.

13. An artificial finger joint comprising the hinge according to claim 1.

14. A container comprising a containing body and a door movable by the hinge according to claim 1.

15. Use of the hinge according to claim 1 in a medical device, and preferably in a medical implant.
